(19) United States
(12) Patent Application Publication
(10) Pub. No.: US 2022/0293253 A1
(43) Pub. Date: Sep. 15, 2022

(54) SYSTEMS AND METHODS USING NATURAL LANGUAGE PROCESSING TO IMPROVE COMPUTER-ASSISTED CODING

(71) Applicant: Intelligent Medical Objects, Inc., Rosemont, IL (US)

(72) Inventors: Brian Bunker, Glenview, IL (US); Joel Graff, Highland Park, IL (US)

(21) Appl. No.: 17/692,054

(22) Filed: Mar. 10, 2022

Related U.S. Application Data
(60) Provisional application No. 63/159,438, filed on Mar. 10, 2021.

(51) Int. Cl. G16H 40/20; G06F 3/04842; G06F 40/284; G06F 40/30; G06F 40/205; G06F 16/242; G06F 16/22; G16H 70/60

(52) U.S. Cl. CPC G16H 40/20; G06F 3/04842; G06F 40/284; G06F 40/30; G06F 40/205; G06F 16/243; G06F 16/22; G16H 70/60

(57) ABSTRACT

Systems and methods for using NLP techniques to improve search engine results for computer-assisted coding. The method includes building a search index for each of the concepts of a health care-related code set, separately or in parallel building a query comprising data from concepts and/or lexicals of a second health care-related code set, pre-processing the data elements derived from each code set, applying a natural language processor to one or both sets of data to generate the search index and the query, inputting the query into a search engine to evaluate the query against the search index, identifying potential matches, presenting the potential matches to the user and receiving a selection of one potential match to be the match, and mapping the concept in the health care-related code set corresponding to the match to the concept and/or lexical of the second health care-related code set corresponding to the query.

### FIELD

Embodiments described herein relate to techniques for improving computer-assisted coding workflows; in particular, for improving natural language processing methods in order to yield more efficient workflows.

### BACKGROUND OF THE INVENTION

Computer-assisted coding (CAC) has been defined historically as the “use of computer software that automatically generates a set of medical codes for review, validation, and use based upon provider clinical documentation.” CAC applications have been researched and implemented to automatically extract and classify terms in a medical document and to assist in the coding of documents. In addition to improving coding productivity, CAC technology can improve coding quality for its users, and it may provide an avenue to improve many documentation- and coding-related processes both within and across organizations.

According to the American Health Information Management Association (AHIMA), CAC can help coders manage the increasing complexity of the workload due to “the increased need for improved data collection and data maintenance as organizations integrate, use, and rely upon more data from disparate data sources.” Bishop J, Bronnert J, Cook J, et al. Automated coding workflow and CAC practice guidance (2013 update). Retrieved Mar. 3, 2021, from https://library.ahima.org/doc?oid=300265#.YEAV7GhKiUk. Other challenges resulting from this increased complexity may include “increased scrutiny for erroneous or fraudulent claims, leaving little tolerance for coding or billing errors” and “advancements in medical care, which require that coding professionals continuously advance their understanding of various clinical subjects such as anatomy, physiology, pathophysiology, and pharmacology,” but even these challenges may be exacerbated by the more technical challenges discussed above. Id.

Within the domain of CAC, there are two different but highly related activities: coding and mapping. Medical coding is focused on identifying the most specific code applicable within the context of an individual patient's care. In comparison, mapping in this context may be defined as the creation of links from concepts or terms in one classification, or ontology, to another for some purpose of data re-use as per the International Standards Organization (ISO). Mapping, as opposed to coding, may facilitate interoperability and may be applied using heuristics and guidelines to support the specific use case. Mapping may not be limited to translation between terminologies or classifications but can be applied to other health data fields in electronic health records. It is based on the philosophy of “code once, use many times.”

Over time, CAC has evolved by incorporating the use of structured text, unstructured text, and natural language processing (NLP). In particular, the advent of NLP techniques has added a new technology to improve knowledge extraction from Electronic Health Records (EHRs), but the use of such NLP techniques and their associated tools also present challenges. For example, early NLP enterprise tools often were too expensive to use for research, leading to free, open-source tools being used instead. Although cheaper, such tools were almost always rules-based and therefore hard to generalize. With the increasing complexity of medical documents, rules-based tools would require a new solution to be created for each use case or domain, increasing the effort and complexity needed to maintain these solutions.

Recent NLP efforts in CAC have focused on extracting concepts from free text and mapping between code sets. Such endeavors further compound the complexity of the problem for several reasons. For example, while complex and voluminous in their own right, EHR or other medical records may be generated using templates, forms, or other methods that result in their data being structured or otherwise stored in some common, shared, and/or recognizable manner. In comparison, free text may not benefit from any such commonality or structuring, so that techniques applicable to a first item or type of free text may not be relevant to a second item or type of such text.

Similarly, code sets continue to grow in number and granularity to the point that even specialized code sets involve a corpus of concepts beyond what an individual can practically maintain.

In this context, terminology or code sets are a set of descriptions used to represent concepts specific to a particular discipline. It also is the foundation of EHR data. For example, the terms “heart attack” and “MI” describe the same concept of myocardial infarction. The concept in turn may be associated with codes that are used for a variety of purposes.

Different healthcare terminologies or code sets may have their own unique features and purposes. For example, one set of terminologies, RxNorm, encodes medications, while another set of terminologies, e.g., Logical Observation Identifiers Names and Codes (referred to under the trademark “LOINC”), is used for laboratory results.

Administrative code sets may be designed to support administrative functions of healthcare, such as reimbursement and other secondary data aggregation. Common examples are the International Classification of Disease (ICD) and the Current Procedural Terminology, which is referred to via the trademark CPT. Each system may be different, e.g., ICD's purpose is to aggregate, group, and classify conditions, whereas CPT is used for reporting medical services and procedures. There are multiple versions of ICD and multiple revisions of those versions. For example, the version of ICD prevalently in use now is ICD-10, which is the 10^{th} revision of ICD. The U.S. version includes modifications for classifying and reporting diseases and is designated as ICD-10-CM (Clinical Modification). Moreover, the 11^{th} version of ICD went into effect in February 2022. In order to appreciate the size and complexity of these code sets, ICD-11 includes over 17,000 unique concepts and more than 120,000 codable terms. ICD-10-CM currently includes more than 72,000 unique codes and is updated periodically to add even more codes, and it is believed that similar modifications will be made to ICD-11 so that the number of unique concepts it includes will continue to grow over time. Although these represent different collections of concepts and related codes, it will be understood unless otherwise discussed separately herein that the disclosure of ICD may represent any or all of these different code sets or revisions thereof, including both past and future revisions.

Clinical code sets have been developed to encode specific clinical entities involved in clinical work flow, such as LOINC and RxNorm. Clinical code sets have been developed to allow for meaningful electronic exchange and aggregation of clinical data for better patient care. For example, sending a laboratory test result using LOINC facilitates the receiving facility's ability to understand the result sent and make appropriate treatment choices based upon the laboratory result. LOINC is similar in size to ICD-10-CM, with more than 70,000 different observation terms.

A reference terminology may be considered a “concept-based, controlled medical terminology.” The Systematized Nomenclature of Medicine Clinical Terms (referred to under the trademark “SNOMED CT”) is an example of this kind of terminology. It maintains a common reference point in the healthcare industry. Reference terminologies also identify relationships between their concepts. SNOMED CT includes concepts such as heart disease and heart valve disorder, and their defined relationship identifies heart valve disorder as a child of heart disease. SNOMED CT is an order of magnitude larger than ICD-10, with more than 350,000 different concepts and relationships among those concepts.

Reference terminology may allow healthcare systems to get value from clinical data coded at the point of care. In general, reference terms may be useful for decision support and aggregate reporting and may be more general than the highly detailed descriptions of actual patient conditions. For example, one patient may have severe calcific aortic stenosis and another might have mild aortic insufficiency; however, a healthcare enterprise might be interested in finding all patients with aortic valve disease. The reference terminology creates links between “medical concepts” that allow these types of data queries.

An important aspect of a well-constructed terminology is concept orientation, typically granular by nature and defined as “a unit of knowledge or thought created by a unique combination of characteristics.” An example of a SNOMED CT concept is aortic valve disorder. A concept may have multiple subconcepts arranged in a hierarchical relationship.

Many clinicians are required to use administrative coding sets (CPT, HCPCS, and ICD-10-CM code sets, etc.) to capture clinical data. However, administrative code sets were designed either to group diagnoses and procedures or to contain broad categories with administrative technical terms with complex rules and guidelines. Examples of this are time durations or vascular branch orders directly stated in various terms.

Administrative codes and terms typically use language that is not natural or familiar for clinicians. For example, in ICD-10-PCS the root operation term “extirpation” is not routinely stated by surgeons. Administrative codes and descriptors also do not contain the different clinical, administrative, and colloquial terms used in healthcare, making it difficult for clinicians, information management professionals, and patients to find the terms they need when performing simple text searches. This disconnect between clinician language and coding sets creates concern over losing clinical intent in the documentation. In addition, forcing a physician to document in administrative terms is uncomfortable and disruptive.

Applicant is a provider of terminology solutions in healthcare, with a core mission to translate from doctors' natural, often semantic-based descriptions of medical concepts to accurate and precise medical terms. To accomplish this task, Applicant has developed and continues to maintain and expand an interface terminology of more than 1.2 million concepts and more than 3.4 million related lexicals, which is centered on a body of over 2.4 million clinician-friendly phrases. By itself, this web of concepts and lexicals is too dense and complex to be fully structured and/or navigated by an individual. Moreover, Appellant also maps the concepts of its interface terminology (either directly or through use of the lexicals mapped to each concept) to the concepts of more than 20 external coding systems and terminologies, increasing the complexity of that mapping and any related NLP efforts in CNC that focus on such mapping, exponentially.

More recently, computer-implemented machine learning (ML) techniques have gained traction as a new technology to assist in automated code generation, knowledge extraction, and classification. The focus of these ML projects was usually narrow, such as for radiology reports, chest x-rays or renal procedures, or they had a limited subset of codes in a given code set. Recently, there have been advances in ML algorithms such as tree-based models, Support Vector Machines (SVMs), and Artificial Neural Networks (ANNs).

Information Retrieval (IR) methods for CAC have received less attention, with studies focused on the comparative performance of different IR algorithms (TF-IDF, BM25F, etc.). The Apache Lucene project is an open-source search library that comprises many such algorithms with additional capabilities.

What are needed are systems and methods that address one or more shortcomings connected with the above discussion.

### SUMMARY OF THE INVENTION

Accordingly, the present disclosure provides systems and methods that overcome one or more of the aforementioned drawbacks by providing new systems and methods for improving computer-assisted coding by using natural language processing coupled with a fuzzy string search and structured data search indexing tool.

In one aspect, a system for using natural language processing to improve computer-assisted coding, the system includes an electronic processor configured to: receive a target code set comprising a plurality of health care-related concepts, the plurality of health care-related concepts comprises at least one thousand health care-related concepts, each concept associated with one or more concept terms and a unique concept code; generate a search index for each concept in the target code set, the search index comprising one or more search terms derived from the one or more concept terms; receive a query, the query comprising one or more health care-related query terms; generate query data derived from the one or more query terms; execute a search query of the query data against the search index; identify one or more matches in the search terms of the search index to the query data; generate, at a user interface, a visual representation of the one or more matches, the visual representation including the one or more concept terms and the unique concept code for the one or more concepts corresponding to the one or more matches; receive a user selection of one of the concepts presented in the visual representation; and generate a map between the query and the selected concept.

In another aspect, a method for using natural language processing to improve computer-assisted coding, the system comprising: receiving, by an electronic processor, a target code set comprising a plurality of health care-related concepts, the plurality of health care-related concepts comprises at least one thousand health care-related concepts, each concept associated with one or more concept terms and a unique concept code; generating, by the electronic processor, a search index for each concept in the target code set, the search index comprising one or more search terms derived from the one or more concept terms; receiving a query, the query comprising one or more health care-related query terms; generating, by the electronic processor, query data derived from the one or more query terms; executing a search query of the query data against the search index; identifying, by the electronic processor, one or more matches in the search terms of the search index to the query data; generating, at a user interface, a visual representation of the one or more matches, the visual representation including the one or more concept terms and the unique concept code for the one or more concepts corresponding to the one or more matches; receiving a user selection of one of the concepts presented in the visual representation; and generating a map between the query and the selected concept.

The foregoing and other aspects and advantages will appear from the following description. In the description, reference is made to the accompanying drawings which form a part hereof, and in which there is shown by way of illustration configurations of the invention. Any such configuration does not necessarily represent the full scope of the invention, however, and reference is made therefore to the claims and herein for interpreting the scope of the invention.

### BRIEF DESCRIPTION OF THE DRAWINGS

FIG. 1 schematically illustrates a method for using natural language processing and fuzzy logic searching via Elasticsearch to improve computer-assisted coding;

FIG. 2 schematically illustrates a visualization of the use of natural language processing techniques to identify potential matches between concepts within multiple code sets;

FIG. 3 schematically illustrates another method for using natural language processing and fuzzy logic searching via Elasticsearch to improve computer-assisted coding; and

FIG. 4 schematically illustrates an example system configurable to using natural language processing and fuzzy logic searching via Elasticsearch to improve computer-assisted coding.

### DETAILED DESCRIPTION

One or more embodiments are described and illustrated in the following description and accompanying drawings. Before any embodiments are explained in detail, it is to be understood the embodiments are not limited in their application to the details of construction and the arrangement of components set forth in the following description or illustrated in the following drawings. Other embodiments are possible, and embodiments described and/or illustrated here are capable of being practiced or of being carried out in various ways. Accordingly, the embodiments described herein may be modified in various ways and other embodiments may exist that are not described herein. Additionally, a component described as performing particular functionality may also perform additional functionality not described herein. For example, a device or structure that is “configured” in a certain way is configured in at least that way but may also be configured in ways that are not listed.

It should also be noted that a plurality of hardware and software-based devices, as well as a plurality of different structural components may be used to implement the invention. In addition, embodiments may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic based aspects of the invention may be implemented in software (for example, stored on non-transitory computer-readable medium) executable by one or more processors. As such, it should be noted that a plurality of hardware and software-based devices, as well as a plurality of different structural components may be utilized to implement various embodiments. It should also be understood that although certain drawings illustrate hardware and software located within particular devices, these depictions are for illustrative purposes only. In some embodiments, the illustrated components may be combined or divided into separate software, firmware, and/or hardware. For example, instead of being located within and performed by a single electronic processor, logic and processing may be distributed among multiple electronic processors. Regardless of how they are combined or divided, hardware and software components may be located on the same computing device or may be distributed among different computing devices connected by one or more networks or other suitable communication links.

As used in the present application, “non-transitory computer-readable medium” comprises all computer-readable media but does not consist of a transitory, propagating signal. Accordingly, non-transitory computer-readable medium may include, for example, a hard disk, a CD-ROM, an optical storage device, a magnetic storage device, a ROM (Read Only Memory), a RAM (Random Access Memory), register memory, a processor cache, or any combination thereof.

In addition, the phraseology and terminology used herein is for the purpose of description and should not be regarded as limiting. For example, the use of “comprising,” “including,” “containing,” “having,” and variations thereof herein is meant to encompass the items listed thereafter and equivalents thereof as well as additional items. Additionally, the terms “connected” and “coupled” are used broadly and encompass both direct and indirect connecting and coupling, and may refer to physical or electrical connections or couplings. Furthermore, the phase “and/or” used with two or more items is intended to cover the items individually and both items together. For example, “a and/or b” is intended to cover: a; b; and a and b.

The present disclosure includes systems and methods to support a mapping process. However, the conceptual framework and benefits are equally applicable to both coding and mapping problems.

In order to better understand the systems and methods of the present disclosure, the following discussion may refer to a specific use case thereof. It will be understood, however, that the systems and methods have applicability well beyond that use case and should not be limited to that use case unless specifically stated so. In particular, in the case described here, a set of interface terminology concept descriptions, previously mapped to ICD-10-CM codes and usable to map an input term to a respective ICD-10-CM code, has been selected to be mapped for the first time to ICD-10 5th edition. One of the challenges associated with this effort is that the 5th edition is only published in physical paper and HTML but not in a curated, structured, electronic format. Thus, the current process for this task has various degrees of technology assistance supporting the effort in order to intake and properly structure the 5th edition concepts to accomplish the desired mapping. Although a complete mapping process may invoke aspects of manual input or review, the disclosure here is focused on the technological aspects involved in that process.

The present system and method may employ multiple data sources in to accomplish the computer-assisted coding described herein. In one example, those data sources may include a base terminology or code set such as Applicant's interface terminology of curated terms and associated data, which may be stored in a database such as a relational database. Given the size and complexity of Applicant's data, elements within that relational database may be stored in a directed graph or similar structure.

A second data source may include a target terminology or code set such as ICD-10 5^{th} edition. As noted above, the second data source may not be available in an electronic format or may be available in an unstructured electronic format such as HTML. Alternatively, the second data source may be stored in a structured data format, although that format may differ from that of the first data source. To utilize the ICD-10 5th edition index, the existing HTML files were digitized by downloading them locally and applying a parsing algorithm that extracted the information into a Python-navigable JSON object. The parsing of certain aspects of the full information contained in the ICD-10 5th edition was left for further development, as it was recognized that such aspects may be useful but not necessarily required for application of the present systems and methods to this particular use case. For example, the information in table form (Neoplasms and Drugs) was not parsed and the “see also” references and textual notes containing coding instructions were not recorded.

A third data source may include a different terminology or code set to which the base terminology or code already may be mapped. In one example, the third code set may be SNOMED CT, although another code set alternatively may be used. The third code set may be internally structured, e.g., via a relational database or through an arrangement of elements such as a hierarchical arrangement. For the purposes for which it is used herein, however, the third data source may be stored as a text file of concepts, and the system may include one or more tables, such as in the relational database storing the base terminology, mapping each of the concepts of the third data source to one or more concepts and/or lexicals of the base code set. Out of the large body of information contained in SNOMED CT, the present systems and methods focused on the map between SNOMED concept identifiers and preferred terms. This content can be accessed in several ways, including a SNOMED browser, a query API, or a fuzzy-search-based API such as the Snowstorm API. Alternatively, the full set of SNOMED CT files may be downloaded, e.g., to an Amazon Web Services (AWS) Simple Storage Service (S3) bucket, and then queried using a query service such as the AWS S3 Select service.

In addition to the input terms themselves, the database already may include human-curated maps between the base code set and a fourth terminology or code set, such as ICD-10-CM. These existing codes may be leveraged as a data source in a mapping algorithm, as discussed in greater detail below. As a clinical modification of the ICD-10 5th edition, the ICD-10-CM codes may be based on a similar structure and provide relevant information, but they are not identical.

The disclosure herein may include a mapping algorithm that, upon receipt of an input term, supplies suggested ICD-10 5^{th} edition codes associated with an index entry. For example, the algorithm may return a ranked list of a plurality of codes, e.g., about 10 codes, along with the associated entries in the ICD-10 5^{th} edition index that led to each suggestion. One such example of suggested codes corresponding to the input term “diastasis of symphysis pubis during delivery” is depicted in Table 1.

TABLE 1
Input
diastasis of symphysis pubis during delivery
Output ICD-10 5^{th} edition Index Entry Code

| | |
|---|---|
| Nonunion \| symphysis pubis, congenital | Q74.2 |
| Delivery (single) \| complicated (by) \| separation, pubic bone | O71.6 |
| (symphysis pubis) | |
| Delivery (single) \| complicated (by) \| placenta, placental \| | O45.9 |
| detachment (premature) | |
| Detachment \| retina (without retinal break) \| serous | H33.2 |
| Delivery (single) \| complicated (by) \| separation, pubic bone | O71.6 |
| (symphysis pubis) | |
| Detachment \| retina (without retinal break) \| with retinal break | H33.0 |
| Detachment \| retina (without retinal break) \| rhegmatogenous | H33.0 |
| Detachment \| retina (without retinal break) \| traction | H33.4 |
| Diastasis \| cranial bones | M84.8 |
| Diastasis \| muscle \| congenital | Q79.8 |

This table may be supplied to a user as part of a user interface that is generated in response to the user inputting the input term into the user input, e.g., via a text input field, a drop-down menu, a selectable portion of the interface such as a window including selectable inputs corresponding to one or more possible inputs, or some other user-input method. As Table 1 may be integrated into a user interface, the interface may include one or more visual indicators for recommending a “correct” or most likely entry within the target terminology or code set. Such visual indicators may include presenting the entry in a visually distinguishable manner such as distinguishing the size, font, and/or color of the text representing the indicator, modifying the text to include bold, italics, underline, or some combination thereof, displaying an arrow or other call-out indicator next to the relevant text, highlighting the background surrounding the relevant text, some combination thereof, or some other form of visual indicator.

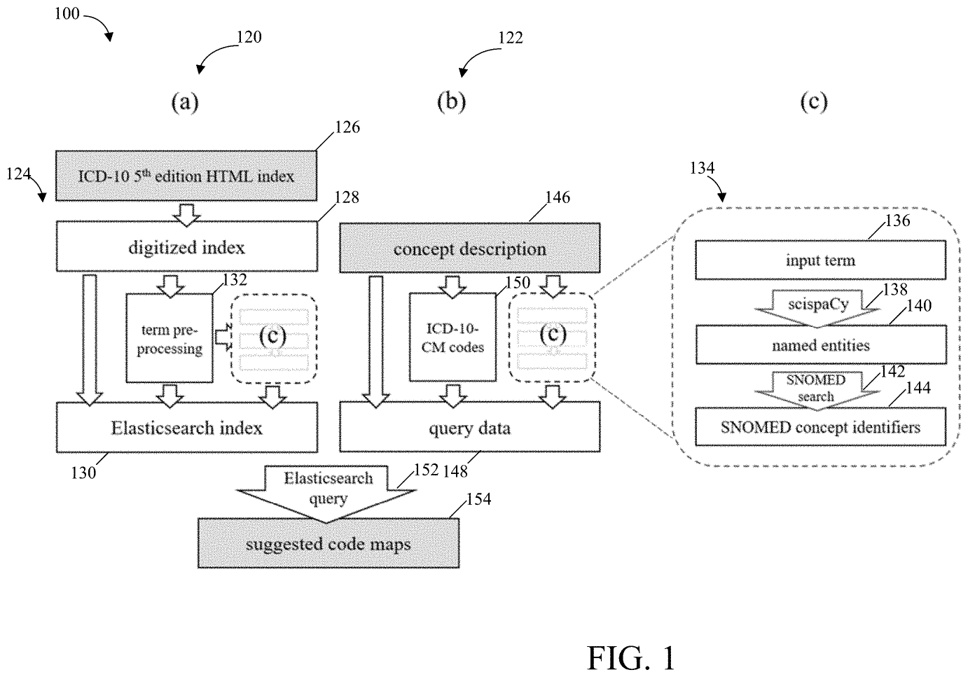

FIG. 1 depicts a high-level flow diagram that illustrates a method 100 taken by the mapping pipeline. Method 100 may include two parallel flows that may follow essentially identical logic. In particular, the method may include a first flow 120 and a second flow 122. First flow 120 may include parsing and processing all entries in a target code set (e.g., ICD-10 5^{th} edition) index, with the resulting information loaded (or “indexed”) to create the reference Elasticsearch index. Second flow 122 may include processing each input term from the base terminology (e.g., an interface terminology) with logic similar to that of first flow 120, with the resulting data being applied to create a query against the Elasticsearch index from first flow 120.

In particular, first flow 120 may include the step 124 of indexing terms of the target code set. As noted above, the target code set may not exist in a structured electronic format but instead may be captured on paper or in an unstructured electronic form such as HTML. In the latter case, the indexing step 124 may include a first step of indexing 126 the unstructured HTML to generate an unstructured electronic index. The method then may include a second step of digitizing 128 the unstructured electronic index so as to create a digitized index. This digitizing step may include ingesting the HTML documents, which may store the underlying target code set data in a different computer-readable format, such as XML, and converting the target code set data into a form that is more easily readable and comprehendible for a user. For example, the results of the digitizing step may include parsed index term words.

Once a digitized index has been created for the target code set, the method may present the user with a plurality of options. First, as seen by the left-most arrow extending downward from the digitizing step 128, the method may include generating 130 an Elasticsearch index, which may include loading any form of the data in the digitized index into one that is usable by the Elasticsearch tool.

Alternatively, at step 132, the method may include pre-processing of one or more terms or types of data prior to generating 130 the Elasticsearch index from that data. The type of pre-processing of the data that occurs may depend on the source and type of data in the target code set. For example, when the target code set is an ICD-10 5th edition code set, terms within that code set may have a hierarchical relationship with one or more other terms or concepts within the code set. For example, the code set may include the term or concept “pregnancy,” which may be related to, as broader than, the term “ectopic.” Pre-processing may be a combinatorial, iterative approach, in which the concepts of the target code set may be broken apart and then reformed into the desired combinations.

In the example provided herein, where the target code set is ICD-10 5th Edition, the ICD-10 5^{th} edition index is originally in HTML format and is digitized at step 128 using a parsing algorithm in Python. The output of the digitization is in a human-readable format such as the output format shown in Table 1. However, while this format may be suitably human readable, it may not be suitable or ideal for an NLP tool (such as the tool discussed below). For example, in the index entry “Delivery (single)|complicated (by)|separation, pubic bone (symphysis pubis),” the terms in parentheses may be optional modifiers. Further, many of the indented terms, separated by the ‘1’ character, may be better assembled backwards instead of forwards. A pre-processing step resolves these issues by creating a one-to-many map from each ICD-10 index entry to a group of possible variations, or string representations. Each string representation flows through subsequent steps and is indexed separately into the Elasticsearch index generated at step 130.

As with the results of the digitizing step 128, in some cases, some or all of the output of the pre-processing step 132 may be used directly at step 130 to generate the Elasticsearch index. Additionally or alternatively, at step 134, one or more NLP techniques may be applied to some or all of the pre-processing output. Details of step 134 are shown in the inset of FIG. 1 with respect to second flow 122, although it should be understood that a similar process applies to step 134 with respect to first flow 120. As seen in FIG. 1, NLP step 134 may include receiving 136 an input term. With respect to first flow 120, the input term may be the term seeking to be mapped to the target code set in its original form or a term output from the pre-processing step 132. At step 138, the input term may be parsed by and run through a natural language processor pipeline so as to generate one or more named entities 140. At step 142, the named entities may be analyzed against terms or concepts within the third data source (e.g., the data source of SNOMED concepts), so as to link one or more third data source (e.g., SNOMED) concept identifiers 144 to each of the named entities generated in step 140, if possible.

The natural language processor identified in FIG. 1, which may be used for named entity recognition, is scispaCy, although it will be appreciated that one or more other NLP tools may be used in step 134. In one instance, scispaCy version 0.2.5, available under an Apache License 2.0, may be used, although it will be appreciated that different versions of scispaCy (or another NLP tool) may be used, particularly as such tools are often updated over time. The spaCy platform is an open-source software library for advanced NLP, widely used across many applications and industries. SciSpacy is built on top of spaCy and operates as a Python package containing spaCy models trained on biomedical and clinical text. Among many relevant features of scispaCy, the present systems and methods may use named entity recognition, for which spaCy requires a specific model. In this case, the model: en_core_sci_lg-0.2.5: “A full spaCy pipeline for biomedical data with a larger vocabulary and 600k word vectors” was used.

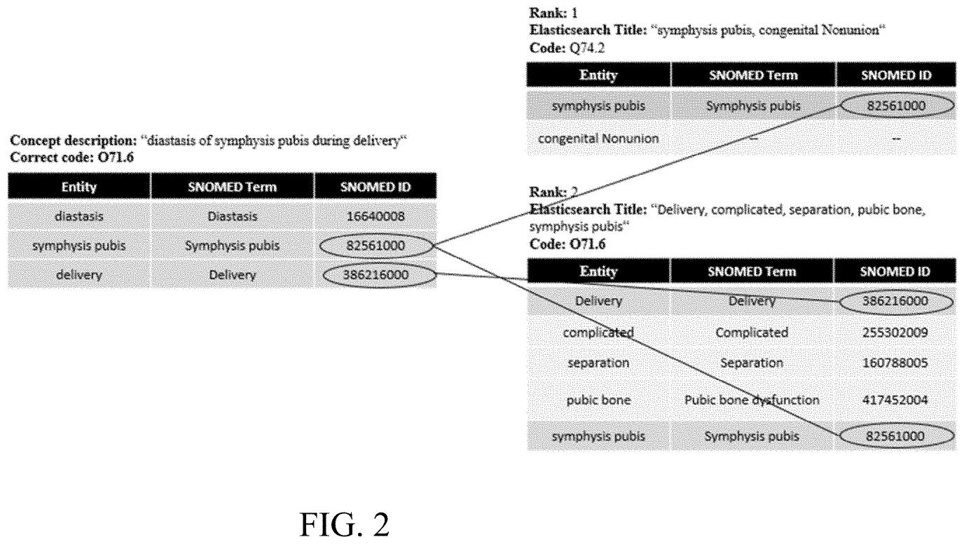

FIG. 2 illustrates an example of the natural language processing techniques of step 134, as applied to the flow 120, for the input term “diastasis of symphysis pubis during delivery.” With regard to first flow 120, (ICD-10 5th Edition) code “O71.6 (Obstetric damage to pelvic joints and ligaments) may have already been identified as mapping to this term in order to verify the effectiveness of the present method. Using scispaCy at step 138, the input term is tagged with three named entities: (“diastasis”, “symphysis pubis”, and “delivery”). Applying the SNOMED search of step 142, it may be determined that these named entities may map to SNOMED concept identifiers (16640008, 82561000, 386216000).

Using Elasticsearch at step 152, the SNOMED IDs are matched to preprocessed ICD-10 5th Edition index entries processed with the same algorithm. The top-ranked match is associated with the ICD-10 5th Edition index entry “Nonunion symphysis pubis, congenital” (code Q74.2) which is incorrect. However, the second-ranked match, to “Delivery (single)|complicated (by)|separation, pubic bone (symphysis pubis)” (code O71.6) is correct.

Returning to FIG. 1, second flow 122 resembles that of first flow 120, although second flow 122 relies on the first data set (e.g., the interface terminology) as its input data set. Within second flow 122, at step 146, the method may receive one or more concept descriptions from the first data set. In the situation where the first dataset is an interface terminology code set, these descriptions may comprise lexicals mapped to the concepts of the interface terminology, rather than the concepts themselves. The lexicals may represent customer terms or collections of user-defined terms so as to capture semantic meaning with respect to the concepts to which they are mapped.

As noted above, the interface terminology may comprise a structured dataset, so it may not be necessary to generate an index of terms from that dataset. Thus, in FIG. 1, the concept descriptions may be used directly at step 148 to generate query data, where the query data comprise the concept descriptions themselves. At step 150, the concept descriptions from the first dataset may be mapped to terms or concepts of a fourth terminology or code set, which in the example of FIG. 1, may be ICD-10-CM, although it will be understood that the fourth code set may be some other code set that can be used for this purpose. The mapping of step 150 may be performed as part of second flow 122. Alternatively, the fourth code set may be pre-mapped to the lexicals of the first code set, so that step 150 may comprise retrieving the relevant mappings for the concept descriptions received at step 146. In either case, the mapped fourth code set concepts then may be used to generate at least some of the query data of step 148.

Staying with second flow 122 of FIG. 1, that process also may include applying NLP techniques at step 134. The details of step 134 with respect to second flow 122 are the same as those discussed above with respect to first flow 120, except that the input terms received at step 136 with respect to second flow 122 are the concept descriptions of the first code set, rather than the pre-processed terms derived from the concepts of the second code set. Thus, following the NLP steps 134 discussed above, the result of those steps may be concepts of the third code set (e.g., SNOMED terms) corresponding to the terms of the first code set that then may be used to generate at least one of the query data of step 148. The query data may be stored as a JSON object or in some other format suitable as an input for the query tool (e.g., Elasticsearch).

First flow 120 and second flow 122 may be run sequentially. Preferably, however, first flow 122 and second flow 124 may be run in parallel with each other. Once each flow has been performed, with the Elasticsearch index generated for the target code set and the query data identified for the first (e.g., interface terminology) code set, the method at step 152 may include executing the Elasticsearch query against those data sets so as to return mapping suggestions 154 for each input term. For each input term, the available information may include the search term itself, the SNOMED concept identifiers, and in most cases a pre-mapped ICD-10-CM code.

Although the first flow 120 and second flow 122 may resemble one another, the first flow 120 may return a plurality of terms for the Elasticsearch index corresponding to each term of the target code set (e.g., ICD-10 5th Edition). In practice, that plurality may actually be thousands or tens of thousands of possible options. For example, as noted above, there are more than 12,000 separate ICD-10 5th Edition codes, and the process may populate the Elasticsearch index with one or more entries corresponding to each of those codes. In comparison, the JSON object storing the query data at step 148 of the second flow 122 may include a single possible options corresponding to the search term and/or the interface terminology lexical.

The query may return a ranked list of suggested ICD-10 5th Edition codes and their associated index entries that best match the search term. Combined query results may include: a string search against the pre-processed ICD-10 5th edition index entry, a search on overlapping SNOMED IDs, ranked by the score: (number of overlapping IDs)/(number of unique combined IDs), and a fuzzy string match on ICD-10-CM versus ICD-10 5th edition code represented as a string.

Elasticsearch is an open-source search platform. One of its capabilities is speed—allowing updates on searches over many documents as the user types. Additionally, it provides very flexible and tunable queries, combining both text and structured data searches with good accuracy. In one instance, the systems and methods may employ Elasticsearch version 7.4, available under an Apache License 2.0, although it will be appreciated that different versions of Elasticsearch (or another search platform) may be used, particularly as such tools are often updated over time.

FIG. 2 illustrates an example of the natural language processing techniques of step 134, as applied to the flows 120 and 122, for the input term “diastasis of symphysis pubis during delivery.” With regard to first flow 120, (ICD-10 5th Edition) code “O71.6 (Obstetric damage to pelvic joints and ligaments) may have already been identified as mapping to this term in order to evaluate the effective ness of the present method. Using scispaCy at step 138, the input term is tagged with three named entities: (“diastasis”, “symphysis pubis”, and “delivery”). Applying the SNOMED search of step 142 to the first flow 120, it may be determined that these named entities may map to SNOMED concept identifiers (16640008, 82561000, 386216000), as represented by the box on the left of FIG. 2.

Similarly, the ICD-10 5th Edition concepts “symphysis pubis, congenital Nonunion” and “deliver, complicated, separation, pubic bone, symphysis pubis” are tagged with the named entities (“symphysis pubis,” and “congenital Nonunion”) and (“delivery,” “complicated,” “separation,” “pubic bone,” and “symphysis pubis”), respectively, as represented by each of the boxes on the right of FIG. 2. FIG. 2 also illustrates that each of these named entities corresponds to a respective SNOMED concept identifier.

Using Elasticsearch at step 152, the SNOMED IDs for the named entities corresponding to the query are evaluated against the SNOMED IDs for preprocessed ICD-10 5th Edition index entries processed with the same algorithm. The top-ranked match is associated with the ICD-10 5th Edition index entry “Nonunion|symphysis pubis, congenital” (code Q74.2) which is incorrect. However, the second-ranked match, to “Delivery (single) complicated (by) separation, pubic bone (symphysis pubis)” (code O71.6) is correct. In this case, the process may evaluate index entries based on a number or a proportion of shared SNOMED IDs in order to determine a strength of match so that, e.g., the index entry with a larger number or greater proportion of shared SNOMED IDs may be ranked higher in the search results than an index entry with a smaller number or proportion of shared SNOMED IDs. It will be appreciated that one or more other heuristics and/or factors may be used in order to rank and return results.

In this example, Elasticsearch was able to identify exact matches between the SNOMED terms/identifiers between the terms in the query (corresponding to an ICD-10-CM lexical) and in the ICD-10 5th Edition, although there was no term in the ICD-10 5th Edition target code set that was a direct match to each of the named entities in ICD-10-CM.

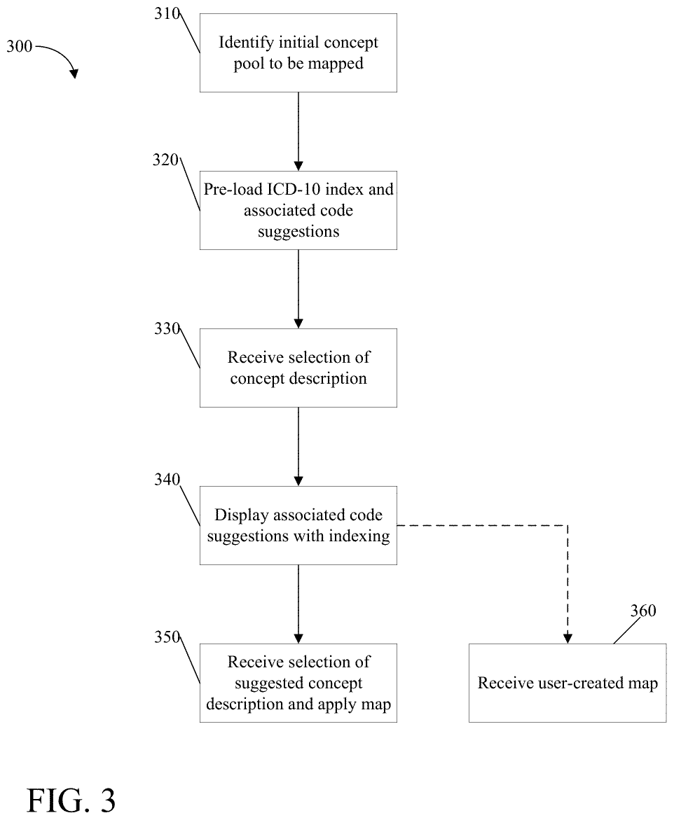

The mapping steps discussed above may be integrated into a full mapping workflow, such as the workflow 300 seen in FIG. 3. The initial concept description pool to be mapped is identified at step 310. At step 320, the ICD-10 index and associated code suggestions are pre-loaded into the platform used by the mapping team. A concept description from ICD-10-CM is selected at step 330, and the associated code suggestions with indexing are displayed at step 340. A mapper then may evaluate the concept description and the accompanying code and index suggestion. At step 350, the system may receive the mapper's selection of the appropriate suggestion for the concept description and then apply the map. Optionally, at step 360, if the appropriate code and index entry are not available in the recommendations, the mapper may utilize their expertise to apply the conventions and guidelines of ICD-10 5^{th} edition and their interpretation of the concept descriptor to apply the correct map. This highlights that there may be situations in the code suggestion process that include a combination of the algorithm results with a human validator to assure the accuracy of the code map.

Validation Statistics

Two types of measures were considered for assessing performance: mapping throughput and accuracy of code suggestions. With regard to the former, one metric may be the throughput in concept descriptions mapped per hour, before and after intervention. Because the time to map each input term was not recorded individually, Table 2 reports the aggregate value for each sample data set without confidence intervals. As Table 2 shows, baseline mapping throughput without the advantage of the method discussed herein was 20 concept descriptions per hour. This leads to a remaining backlog amounting to thousands of hours of future effort. In comparison, a 44.5% increase in mapping throughput was achieved after applying the techniques disclosed herein.

TABLE 2
Sample Data

| | |
|---|---|
| training set: in-sample | n = 613 |
| training set: out-of-sample | n = 1000 |

Throughput

| | |
|---|---|
| pre-study | 20.0/hour |
| post-study | 28.9/hour |
| throughput improvement | 44.5% |

Mapping Accuracy

| | In-Sample | Out-Of-Sample |
|---|---|---|
| A₁: fraction of cases with correct | 0.495 | 0.525 |
| suggestion in rank 1 of search | (0.040) | (0.031) |
| results. | | |
| A₁₀: fraction of cases with correct | 0.668 | 0.689 |
| suggestion within top 10 search | (0.037) | (0.029) |
| results. | | |
| MRR: mean reciprocal rank. | 0.544 | 0.577 |
| | (0.019) | (0.015) |

As noted above, a secondary metric is the accuracy of the code suggestions. The goal is to display the correct code and its associated index entry as prominently as possible within the search results. This is essentially a search application, and metrics including mean reciprocal rank (MRR), precision-at-k (p@k), and discounted cumulative gain (DCG) are available to evaluate search performance. However, some of these metrics are normally used to evaluate search results for a single input, rather than an aggregate over a large sample of searches, and they may not fully capture the present objectives. For example, p@k and DCG reward search results with multiple correct codes, whereas a concern here may primarily be to return the correct code and associated index entry at least once.

Table 2 reports the following statistics: A1 (the fraction of terms for which the correct code was displayed as the first search result); A10 (the fraction of terms for which the correct code was displayed within the first 10 search results); and the mean reciprocal rank (MRR).

The measures A1 and A10 are averages over Bernoulli trials, with large sample size and a success rate not close to 0 or 1. Therefore the normal approximation interval is reliable, expressed as:

$$\hat{p} \pm z \sqrt{\frac{\hat{p} ( 1 - \hat{p} )}{n}}$$

where \hat{p} is the estimated fraction of successes—i.e., the value of A1 and A10 respectively—and z is the (1−α/2) quantile of the standard normal distribution. For a 95% confidence interval, z=1.96.

A popular performance metric for information retrieval applications is the mean reciprocal rank. The mean reciprocal rank is defined as:

$$MRR = \frac{1}{n} {\sum }_{i = 1}^{n} \frac{1}{{rank}_{i}}$$

where n is the number of search terms and rank, is the rank of the first correct answer in the search results for the ith term. If a search does not return any results, rank, is considered infinite and the reciprocal rank is zero.

There is no well-defined probability distribution for the reciprocal rank. However, MRR is in the form of a sum over independent trials. Assuming the distribution is the same across trials, we can apply the Central Limit Theorem to obtain confidence intervals. Let X be a variable with any distribution sampled n times and let μ and σ be the true mean and standard deviation of X. If n is large enough, then the distribution of (X⁻−μ)/σ is approximately a standard normal distribution if n is large. This leads to a confidence interval (MRR−s/\sqrt{n},MRR+s/\sqrt{n}), where s is the sample standard deviation of the reciprocal rank.

Note that the criterion for success is a correct code match, without regard to whether the ICD-10 index entry correctly matched the input term. There is a many-to-one map from ICD-10 index entry to ICD-10 code, leading to occasional instances where the index entry would be regarded as incorrect by a coding expert, but the match is still considered a success in this study.

The present techniques yield a mapping accuracy of 0.525 out-of-sample for the top-ranked code suggestion, and a 0.689 accuracy for the correct answer to be displayed within the top 10 code suggestions.

The out-of-sample MRR for this study was 0.577 (0.015) indicating that the correct map was displayed on average at the second rank or higher.

Without additional model tuning, the findings led the team to determine correct code maps with accuracy comparable to other successful CAC algorithms. Mapping accuracy was slightly improved in the out-of-sample data. Thus, the present system and method may significantly approve the efficiency and throughput of previous CAC processes without having to make a trade-off on accuracy.

This mapping application was performed without the benefit of medical records or patient-specific context. The mapping process relies upon the specific guidance and conventions of the target code set and/or terminology to apply the most appropriate map for the concept. The aim is to create a pipeline that is generalized enough to be able to handle all areas of ICD-10 5th Edition, and possibly additional code sets in the future.

Some of the choices for components of the algorithm, in particular various solutions for mapping of entities to SNOMED and the mapping of SNOMED to ICD-10, are in the public domain.

For example, a popular choice for target entities in named entity recognition and entity linking tasks is the US National Library of Medicine Unified Medical Language System (UMLS) concepts. SciSpacy offers UMLS concept linking, which may be explored during future enhancement to the mapping pipeline.

Further, crosswalk mapping directly from SNOMED CT concepts to ICD-10 5th edition codes is publicly available, although the map is not intended to provide complete automation of ICD-10 from SNOMED CT. A crosswalk is defined as “lists of translating codes from one system to another.” The use of an external crosswalk was not considered in this study because the map from initial concept description to ICD-10 term involves multiple SNOMED concept IDs from both source and target. The concept descriptions in Applicant's interface terminology generally have a high level of detail not captured in a single SNOMED code, and the same is true for ICD-10 index entries. Moreover, crosswalks are not universally applied across applications and contain rules and assumptions which are not transferable. Additionally, Applicant explored techniques incorporating such additional components. While not detrimental to the methods set forth herein, it was verified that those components had little effect on the results.

A method for computer-assisted coding was developed for a specific case involving the mapping of a large collection of concept descriptions to ICD-10 5th edition codes. The use case is representative of many practical situations where an NLP or machine learning task must be prepared with a small project-specific dataset. Moreover, while CAC is essentially a classification task, the possible classes greatly outnumber the samples in the training set. These considerations preclude the use of deep learning algorithms and indeed most machine learning algorithms.

As an alternative, a mapping algorithm was constructed using an open-source NLP and named entity recognition platform (scispaCy), combined with the Elasticsearch search engine. Most of the configuration for the unique data set could be accomplished within the Elasticsearch query or else within simple pre-processing code. Although the search engine discussed herein and used in the specific use cases described herein is Elasticsearch, it will be appreciated that the systems and methods disclosed herein may not be limited to use of just that engine and that other search and analytics engines may be used instead.

The present systems and methods have the virtue of simplicity and versatility, so that they can be applied to similar use cases with little overhead.

While code mapping is essentially a classification task, the number of classes is much larger than any possible training set. For instance, the ICD-10 5^{th} edition contains over 12,000 codes. Further, the kind of information available to create features was unique to the project: i.e., we already had the ICD-10-CM codes.

With these considerations in mind, the present system avoided a standard machine learning architecture requiring specific modeling on the feature set or demanding a large training set, while still achieving the accuracy and throughput results discussed above. Elasticsearch was employed as an alternative that can be readily adapted to new applications simply by modifying the query.

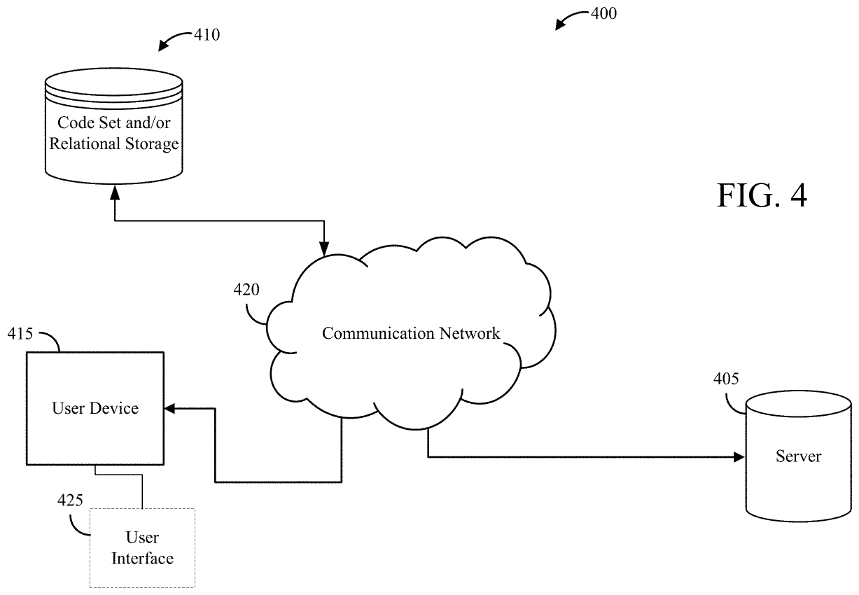

FIG. 4 illustrates a system 400 for using natural language processing and Elasticsearch to improve computer-assisted coding workflow according to some embodiments. As illustrated in FIG. 4, the system 400 includes a server 405, an electronic code set and/or relational storage 410 storing one or more of the code sets disclosed herein, relationships between entities within one or more of those code sets, and/or relationships between entities of multiple code sets, and a user device 415. In some embodiments, the system 400 includes fewer, additional, or different components than illustrated in FIG. 4. For example, the system 400 may include multiple servers 405, multiple storage devices 410, multiple user devices 415, or a combination thereof. Also, in some embodiments, one or more components of the system 400 may be combined into a single component. As one example, the code set and/or relational storage 410 may be included in the server 405. Alternatively or in addition, in some embodiments, the functionality (or a portion thereof) described as being performed by a component of the system 400 may be distributed among multiple components.

The server 405, the code set and/or relational storage 410, and the user device 415 communicate over one or more wired or wireless communication networks 420. Portions of the communication networks 420 may be implemented using a wide area network, such as the Internet, a local area network, such as Bluetooth™ network or Wi-Fi, and combinations or derivatives thereof. It should be understood that in some embodiments, additional communication networks may be used to allow one or more components of the system 400 to communicate. Also, in some embodiments, components of the system 400 may communicate directly as compared to through a communication network 420 and, in some embodiments, the components of the system 400 may communicate through one or more intermediary devices not shown in FIG. 4.

The server 405 includes a computing device, such as a server, a database, or the like. The server 405 includes an electronic processor (for example, a microprocessor, an application-specific integrated circuit (ASIC), or another suitable electronic device), a memory (for example, a non-transitory, computer-readable medium), and a communication interface. The electronic processor, the memory, and the communication interface communicate wirelessly, over one or more communication lines or buses, or a combination thereof. It should be understood that the server 405 may include additional components and may perform additional functionality than the functionality described herein. For example, in some embodiments, the functionality described herein as being performed by the server 405 may be distributed among servers or devices (including as part of services offered through a cloud service), may be performed by one or more user devices 415, or a combination thereof.

The communication interface allows the server 405 to communicate with devices external to the server 405. For example, as illustrated in FIG. 4, the server 405 may communicate with the electronic code set and/or relational storage 410, the user device 415, or a combination thereof through the communication interface. The communication interface may include a port for receiving a wired connection to an external device (for example, a universal serial bus (“USB”) cable and the like), a transceiver for establishing a wireless connection to an external device (for example, over one or more communication networks, such as the Internet, local area network (“LAN”), a wide area network (“WAN”), and the like), or a combination thereof.

The electronic processor is configured to access and execute computer-readable instructions (“software”) stored in the memory. The software may include firmware, one or more applications, program data, filters, rules, one or more program modules, and other executable instructions. For example, the software may include instructions and associated data for performing a set of functions, including the methods described herein.

The user device 415 is a computing device and may include a desktop computer, a terminal, a workstation, a laptop computer, a tablet computer, a smart watch or other wearable, a smart television or whiteboard, or the like. Although not illustrated in FIG. 4, the user device 415 may include similar components as the server 405, such as electronic processor (for example, a microprocessor, an application-specific integrated circuit (ASIC), or another suitable electronic device), a memory (for example, a non-transitory, computer-readable storage medium), a communication interface, such as a transceiver, for communicating over the communication network 420 and, optionally, one or more additional communication networks or connections, and one or more human machine interfaces. For example, to communicate with the server 405, the user device 415 may store a browser application or a dedicated software application executable by an electronic processor. In one aspect, the system 400 is described herein as developing and implementing methods for using natural language processing and Elasticsearch to improve computer-assisted coding through the server 405. However, in other aspects, the functionality described herein as being performed by the server 405 may be locally performed by the user device 415. For example, in some embodiments, the user device 415 may store the application.

A user may use the user device 415 to interact with, e.g., the application 430. Accordingly, in some embodiments, a user may use the user device 415 to interact with the application to perform the workflow (or a portion thereof) of FIG. 1 and/or FIG. 3. In order to facilitate this interaction, the system 400 may transmit instructions to the user device 415 to generate and/or display a user interface 425, with the user interface 425 including data input features such as text boxes, drop-down or other selectable menus, radio buttons, etc., for receiving user inputs to undertake the first and/or second flows 120, 122. The user interface also may generate an interactive region in response to the user providing a search or target phrase. The interactive region may permit a user to select one of the suggested ICD-10 5^{th} Edition terms as corresponding to the input term, so that selecting a term causes the search query to be mapped to the term. In one aspect, the interactive region may include the ranked list of suggested code maps. For example, the interactive region may include a region resembling Table 1 discussed above.

The user interface may include a cue of some kind to visually distinguish a “best,” “most accurate,” and/or “most recommended” code map suggestion from other results being presented. As seen in Table 1, the visual cue may include changing a font effect (e.g., bold face, underlining, or italicizing). Additionally or alternatively, the visual cue may include highlighting a region surrounding the recommended suggestion, generating a distinctive icon before, alongside, or after the recommended result.

The present disclosure generates a search index for a target code set such as ICD-10 5th Edition to be queried by a search engine such as Elasticsearch to identify a match in that code set to a concept description (e.g., a lexical) that corresponds to a concept in a second code set such as an interface terminology. As described above, the second code set may be significantly more granular (e.g., an order of magnitude or more) than the target code set, so that there may be a one-to-many relationship between concepts of the target code set and a concept description in the second code set. In another aspect, the search index may be generated from the concepts or concept descriptions of the second code set, and the query may be derived from a concept of the target code set. Because there are fewer concepts in the target code set than there are concepts or concept descriptions in the second code set, this configuration may require even fewer queries in order to generate a complete map between the target code set and the second code set. In this alternative configuration, it should be understood that there then may be multiple “correct” results in the search index, reflecting that there may be a many-to-one relationship between the concepts or concept descriptions of the second code set, e.g., the interface terminology, and the target code set.

The embodiments described herein have been described in terms of one or more preferred configurations, and it should be appreciated that many equivalents, alternatives, variations, and modifications, aside from those expressly stated, are possible and within the scope of the invention.

1. A system for using natural language processing to improve computer-assisted coding, the system comprising:
an electronic processor configured to
receive a target code set comprising a plurality of health care-related concepts, the plurality of health care-related concepts comprises at least one thousand health care-related concepts, each concept associated with one or more concept terms and a unique concept code;
generate a search index for each concept in the target code set, the search index comprising one or more search terms derived from the one or more concept terms;
receive a query, the query comprising one or more health care-related query terms;
generate query data derived from the one or more query terms;
execute a search query of the query data against the search index;
identify one or more matches in the search terms of the search index to the query data;
generate, at a user interface, a visual representation of the one or more matches, the visual representation including the one or more concept terms and the unique concept code for the one or more concepts corresponding to the one or more matches;
receive a user selection of one of the concepts presented in the visual representation; and
generate a map between the query and the selected concept.

2. The system of claim 1, wherein the electronic processor being configured to generate a search index comprises the processor being configured to parse, for each concept of the plurality of concepts, the associated one or more concept terms into a plurality of separate terms.

3. The system of claim 2, wherein the electronic processor being configured to generate a search index comprises the processor being configured to apply a natural language processor (NLP) to each concept in order to derive the one or more search terms.

4. The system of claim 3, wherein the electronic processor being configured to generate a search index further comprises the processor being configured to evaluate the NLP-derived one or more search terms against concepts of a third code set, each concept of the third code set comprising one or more third code set concept terms and a unique third code set concept code.

5. The system of claim 1, wherein the query comprises a lexical of a health care-related interface terminology code set, the lexical comprising a semantic expression of a concept of the interface terminology code set, wherein the lexical or its associated concept is mapped to a concept of a fourth code set, and wherein the electronic processor being configured to generate query data comprises the processor being configured generate query data from the mapped concept of the fourth code set.

6. The system of claim 5, wherein the electronic processor being configured to generate query data comprises the processor configured to apply a natural language processor (NLP) to the mapped concept of the fourth code set to derive the query data.

7. The system of claim 6, wherein the electronic processor being configured to generate query data comprises the processor being configured to evaluate the NLP-derived query data against concepts of a third code set, each concept of the third code set comprising one or more third code set concept terms and a unique third code set concept code to derive a query-related concept of the third code set.

8. The system of claim 7, wherein the electronic processor being configured to generate a search index comprises the processor being configured to apply a natural language processor (NLP) to each concept in order to derive the one or more search terms and to evaluate the NLP-derived one or more search terms against concepts of the third code set, and wherein the processor being configured to identify one or more matches in the search terms to the query data comprises the processor executing a search engine to evaluate the concepts of the third code set corresponding to the NLP-derived one or more search terms against the query-related concept of the third code set.

9. The system of claim 7, wherein the third code set is the Systematized Nomenclature of Medicine Clinical Terms.

10. The system of claim 1, wherein the target code set is an International Classification of Disease code set.

11. A method for using natural language processing to improve computer-assisted coding, comprising:
receiving, by an electronic processor, a target code set comprising a plurality of health care-related concepts, the plurality of health care-related concepts comprises at least one thousand health care-related concepts, each concept associated with one or more concept terms and a unique concept code;
generating, by the electronic processor, a search index for each concept in the target code set, the search index comprising one or more search terms derived from the one or more concept terms;
receiving a query, the query comprising one or more health care-related query terms;
generating, by the electronic processor, query data derived from the one or more query terms;
executing a search query of the query data against the search index;
identifying, by the electronic processor, one or more matches in the search terms of the search index to the query data;
generating, at a user interface, a visual representation of the one or more matches, the visual representation including the one or more concept terms and the unique concept code for the one or more concepts corresponding to the one or more matches;
receiving a user selection of one of the concepts presented in the visual representation; and
generating a map between the query and the selected concept.

12. The method of claim 11, wherein generating a search index comprises parsing, for each concept of the plurality of concepts, the associated one or more concept terms into a plurality of separate terms.

13. The method of claim 12, wherein generating a search index comprises applying, by the processor, a natural language processor (NLP) to each concept in order to derive the one or more search terms.

14. The method of claim 13, wherein generating a search index further comprises evaluating the NLP-derived one or more search terms against concepts of a third code set, each concept of the third code set comprising one or more third code set concept terms and a unique third code set concept code.

15. The method of claim 11, wherein the query comprises a lexical of a health care-related interface terminology code set, the lexical comprising a semantic expression of a concept of the interface terminology code set, wherein the lexical or its associated concept is mapped to a concept of a fourth code set, and wherein generating query data comprises generating query data from the mapped concept of the fourth code set.

16. The method of claim 15, wherein generating query data comprises the processor applying a natural language processor (NLP) to the mapped concept of the fourth code set to derive the query data.

17. The method of claim 16, wherein generating query data comprises evaluating the NLP-derived query data against concepts of a third code set, each concept of the third code set comprising one or more third code set concept terms and a unique third code set concept code to derive a query-related concept of the third code set.

18. The method of claim 17, wherein generating a search index comprises applying a natural language processor (NLP) to each concept in order to derive the one or more search terms and evaluating the NLP-derived one or more search terms against concepts of the third code set, and wherein identifying one or more matches in the search terms to the query data comprises executing, by the processor, a search engine to evaluate the concepts of the third code set corresponding to the NLP-derived one or more search terms against the query-related concept of the third code set.

19. The method of claim 17, wherein the third code set is the Systematized Nomenclature of Medicine Clinical Terms.

20. The method of claim 11, wherein the target code set is an International Classification of Disease code set.
